(19) United States
(12) Patent Application Publication
(10) Pub. No.: US 2023/0390536 A1
(43) Pub. Date: Dec. 7, 2023

(54) PRESSURE INDUCED DEFLATABLE FOLEY'S CATHETER

(71) Applicants: K. NAGAJYOTHI, Tirupati (IN); B. SURESH, Tirupati (IN)

(72) Inventor: B. SURESH, Tirupati (IN)

(21) Appl. No.: 18/253,007

(22) PCT Filed: Nov. 13, 2021
(86) PCT No.: PCT/IB2021/060533

§ 371 Date: May 15, 2023

(30) Foreign Application Priority Data
Nov. 13, 2020 (IN) 202041020112

(51) Int. Cl. A61M 25/10; A61M 25/00

(52) U.S. Cl. CPC A61M 25/1002; A61M 25/0017; A61M 2025/1084; A61M 25/10185; A61M 25/1011

(57) ABSTRACT

A catheter (1) is disclosed which has a primary balloon (2) to be inflated or deflated, a pumping means (3) for pumping a working fluid into and out of the primary balloon (2) to inflate or deflate it, and a restraining mechanism (4) to keep a first part (5) of the primary balloon (2) or an opening (7) of the primary balloon (2) in a restrained position until a pressure inside the primary balloon (2) is within a predefined threshold and in an expanded position when the pressure inside the primary balloon (2) surpasses the predefined threshold and until the pressure inside the primary balloon (2) gets below the predefined threshold. This helps in retaining the working fluid inside the primary balloon (2) until the pressure is within the threshold, and to move it out of the balloon when it is beyond the threshold.

### FIELD OF INVENTION

The invention relates to catheters which are put inside a human body part for a particular purpose. More specifically, the invention relates to catheters which use balloons to implement functionality to the catheter. In furtherance, the invention relates to making balloon-based catheters safe while operating or using the catheters inside the human body part.

### BACKGROUND

Foley's catheter is used commonly in urological procedures and also in almost all patients admitted into an Intensive care unit. Foley's catheter (named for Frederic Foley, who produced the original design in 1929) is a flexible tube that a clinician passes through the urethra and into the bladder to drain urine. It is the most common type of indwelling urinary catheter. The tube has two separated channels, or lumens, running down its length.

Indwelling urinary catheters are most commonly used to assist people who cannot urinate on their own. Indications for using a catheter include providing relief when there is urinary retention, monitoring urine output for critically ill persons, managing urination during surgery, and providing end-of-life care.

Foley catheters are used during the following situations:
On patients who are anesthetized or sedated for surgery or other medical care.
On comatose patients.
On some incontinent patients.
On patients whose prostate is enlarged to the point that urine flow from the bladder is cut off.
On patients with acute urinary retention.
On patients who are unable to void urine due to paralysis or physical injury to the urethra.
Following urethral surgeries.
On patients with kidney disease whose urine output must be constantly and accurately measured/monitored.
Before and after cesarean section.
Before and after hysterectomy.
On patients who have had genital injury.
On anorexic patients who are unable to use standard toilets due to physical weakness and whose urine output must be constantly measured/monitored.

There are several risks in using a Foley's catheter (or catheters generally), including:
The balloon can break as the healthcare provider inserts the catheter. In this case, all balloon fragments must be removed preferably endoscopically.
Urine stops flowing into the bag due to urethral placement of the catheter or even when the catheter is correctly placed into the urinary bladder due to its thick contents which are difficult to drain (pus/blood clot(s)/chyle).
Catheter can be blocked due to several reasons which should be flushed or replaced.
Urethra begins to bleed due to several causes including catheter induced urethral trauma, forceful negotiation of the catheter in a patient with stricture urethra and also in patients on antiplatelet agents.
Catheter associated UTI (CAUTI).
Defective catheters may be supplied, which break in situ. The most common fractures occur near the distal end or at the balloon.
The balloon might not inflate/inflate with difficulty associated with/without urethral pain when inflated in the urethra leading to urethral injury and attendant complications (i.e., when the balloon is inflated before the Foley's catheter is completely inserted into the urinary bladder) like bleeding, damage or even rupture of the urethra leading to the long-term permanent scarring and urethral stricture formation in some individuals.
Catheters can be pulled out by patients while the balloon is still inflated, leading to major complications or even death. This may occur when patients are mentally impaired (e.g. they have Alzheimer's) or are in a mentally altered state (e.g. they are coming out of surgery).
In few cases of Foley's catheterization, the balloon couldn't be deflated due to clogging of the channel connecting the balloon to the inflation port and thus resulting in failure to remove the Foley's catheter. In such cases the removal involves either by suprapubic balloon rupture with a spinal needle/PNL needle (using ultrasound guidance in the radiology suite) or by endoscopic rupture of the balloon and removal the residual balloon fragments and the catheter in the OR (operating room).
In some patients, catheter couldn't be negotiated into the urinary bladder despite adequate urethral lumen resulting from causes like tortuous or deformed urethra, urethral diverticulae, false passages in the urethra secondary to prior urological intervention, and rarely passable multiple urethral stricturous rings. In these situations, catheter could be negotiated into bladder by back loading the Foley's catheter over an appropriate guide wire but without which may result in urethral trauma.

It is to be noted that the similar issues and safety related problems may occur while using any balloon-based catheters which are used for any other functionality like installing a medical device or stent inside a blood vessel.

There were few attempts made to handle the safety related problems in balloon-based catheters. They are listed below.

US Publication No. U.S. Pat. No. 9,084,868B2 discloses various embodiments of catheter which solves safety related problems related to excess pressure built up inside the retention balloon which can lead to patients' discomfort or even leads to damaging the bladder or urinary tract while incorrectly installing the catheter or while incorrectly removing the catheter or due to their issues. One embodiment mentions providing a burstable region between lumens for fluid inlet and outlet, and retention balloon filling inlet. Another embodiment mentions about, provides burstable region between the retention balloon filling inlet and external environment. Yet another embodiment mentions a placement of safety balloon in the retention balloon filling inlet checked by a lock so as to prevent filling of the safety balloon till a pressure is built up. The placement of this safety balloon is provided such that it is outside the human body when the catheter is installed, and its purpose is to be a visual indicator to the Doctor, so that he can monitor the situation and handle the catheter accordingly. In another embodiment, the slit valve is provided between lumens for fluid inlet and outlet, and retention balloon filling inlet. The slit valve opens up beyond a pressure limit. The embodiments mentioned in this prior art has lacunas, which either makes the catheter further unusable or requires skills to monitor during pressure built up.

US Publication No. U.S. Pat. No. 9,055,949B2 mentions an implantable device is provided for controlling hemorrhage in a body cavity, comprising an expandable balloon and a conduit for supplying a physiologically compatible fluid to inflate the balloon. When the balloon tamponade device is implanted or inserted into the body cavity, it is inflated with a physiologically suitable fluid, so that the balloon generally conforms to the body cavity and exerts compressive force against the walls, tissues or structures of the body cavity to control hemorrhage. The balloon may have a deforming means to limit expansion of the balloon in a direction to facilitate expansion of the balloon in another direction. The device may have additional tubes within the conduit, or a plurality of separate lumens within the conduit or tubes to allow drainage and irrigation to the body cavity. There is also provided a cuff for attachment of an external traction to the balloon tamponade, to facilitate the compressive effect of the device. The device mentioned here do not have any mechanisms for safety, as the inflation procedures are limited to control hemorrhage, and accordingly balloon deforming means and cuffs are provided for changing direction of expansion of balloon.

US Publication No. US2010/0217189A1 mentions a pressure relief apparatus for a balloon dilation catheter having a shaft with a dilation balloon attached to the distal end of the shaft and an inflation/deflation lumen for inflating and deflating the balloon includes a pressure relief port formed through the wall of the inflation/deflation lumen with a pressure relief member secured across the pressure relief port to form a fluid tight seal such that the fluid tight seal formed by the pressure relief member fails at a predetermined pressure to release pressure from the inflation/deflation lumen through the pressure relief port. This embodiment also provides for the catheter which becomes unusable after failing of pressure relief member.

US Publication No. US2019/0151628A1 mentions a balloon catheter having an elongated catheter shaft defining a fluid drainage lumen and a balloon inflation lumen. The balloon catheter includes a fluid drainage port disposed about the distal end of the catheter shaft in fluid communication with the fluid drainage lumen, and a balloon inflation port disposed about the distal end of the catheter shaft in fluid communication with the balloon inflation lumen. A balloon portion is disposed about the distal end of the catheter shaft in fluid communication with the balloon inflation port. A release device is disposed in fluid communication with the balloon portion and the fluid drainage lumen, and includes an activating member. A tether is attached to the activating member of the release device. Tension applied to the tether activates the release device, enabling fluid flow from the balloon portion into the fluid drainage lumen and out of the body. This device has a lacuna, as it is not self-activated, rather a tension is required to be externally employed for enabling the release of pressure and require regular administration.

US Publication No. US2006/0167438A1 mentions a pressure-limiting or breakaway catheter includes a multi-lumen shaft having a distal end and a hollow balloon portion disposed at the distal end of the shaft. The balloon portion has a distal end and an interior. The shaft and/or the balloon portion has a balloon safety valve formed to open to the environment outside the shaft and/or the balloon portion when greater than a given bursting pressure exists within the shaft and/or the interior of the balloon portion. The safety valve can be formed to burst at a first breaking force less than a second breaking force required to burst the balloon portion and/or the shaft. Also provided is a breakaway catheter kit including a set of the breakaway catheters, each having the balloon safety valve with different safety valve breaking constants. This catheter also has a lacuna, as part of the safety mechanism the safety valve bursts, and makes the catheter unusable.

Hence, there is a need to make such balloon-based catheters which are safe while operating or installing inside a human body part, and these safety features should not hamper reusability of these catheters, and also should not require continuous monitoring of the catheters.

### Object of the Invention

The object of the invention is to provide a mechanism to make usage of a balloon-based catheter safe while operating, using or installing the catheter inside a human body part, such as urinary bladder, urethra or a blood vessel.

### SUMMARY OF THE INVENTION

The object of the invention is achieved using a catheter as disclosed further. The catheter includes a primary balloon which is inflated or deflated to retain the primary balloon inside a human body part or to apply a medication inside the human body part, or to install a device inside the human body part. The catheter further includes inflation/deflation port which can be connected to a pumping means that pumps in a fluid to inflate the primary balloon, and to pump out the fluid to deflate the primary balloon. The catheter also includes a restraining mechanism which keeps a first part of the primary balloon or an opening of the primary balloon in a restrained position until a pressure inside the second part of the primary balloon or the primary balloon is within a first predefined threshold, so as to close a fluid communication between a second part of the primary balloon and the first part of the primary balloon, or between the opening of the primary balloon to an environment outside of the primary balloon. Further, the restraining mechanism keeps the first part of the primary balloon or the opening of the primary balloon in an expanded position when the pressure inside the second part of the primary balloon or the primary balloon surpasses the first predefined threshold and until the pressure inside the second part of the primary balloon or the primary balloon gets below the first predefined threshold, so as to open a fluid communication between the second part of the primary balloon and the first part of the primary balloon, or between the opening of the primary balloon to an environment outside of the primary balloon.

According to one embodiment of the catheter, wherein the restraining mechanism is a stretchable band which covers a part of the primary balloon dividing the primary balloon into the first part and the second part, such that the second part is in direct fluid communication with the pumping means, or is placed at an opening of the primary balloon which connects the primary balloon to the environment outside of the balloon. This embodiment provides an efficient implementation of the restraining mechanism.

According to another embodiment of the catheter, wherein the first part of the primary balloon comprises a weak area which bursts when a pressure inside the first part of the primary balloon surpasses a second predefined threshold. This embodiment is helpful, as it further checks on the safety feature itself provided through the first part of the primary balloon. In case the first part moves into the red alert pressure zone, the weak area bursts to control the excess built up pressure.

According to yet another embodiment of the catheter, wherein the first part of the primary balloon and the environment outside of the primary balloon are fluidly connected to each other through a fluid communication channel. The catheter includes a flow controller which is adapted to open the fluid communication channel between the first part of the primary balloon and the environment outside of the primary balloon when a pressure inside the first part of the primary balloon surpasses the second predefined threshold, and to close the fluid communication channel between the first part of the primary balloon and the environment outside of the primary balloon when the pressure inside the first part of the balloon gets below the second predefined threshold. This embodiment provides another way to check the safety features provided through the first part of balloon, where the excess pressure is released to outside environment through the flow controller. This feature makes the catheter reusable with the safety feature still intact.

According to one embodiment of the catheter, wherein the flow controller is an automated valve or a retractable plug. The automated valve moves on or off the fluid communication channel for opening or closing the fluid communication channel. The retractable plug unplugs itself from the fluid communication channel or retract back to plug itself onto the fluid communication channel for opening or closing the fluid communication channel. The automated valve or the retractable plug works on at least one of the mechanical, electrical or magnetic principles, or combination thereof. This embodiment provides for various mechanisms to implement the flow controller efficiently.

According to another embodiment of the catheter, wherein the environment outside the primary balloon is a working fluid channel through which the primary balloon is inflated or deflated through the inflation/deflation port using a working fluid, or a body fluid drainage channel through which a body fluid is drained out of the human body, or lumen/cavity of the body part in which the primary balloon of the catheter is placed, or a secondary balloon connected to the primary balloon through a fluid communication channel via the restraining mechanism. The secondary balloon is placed in proximity to the primary balloon. This embodiment provides for various possible ways to implement the safety mechanism of the primary balloon and environment outside the primary balloon for venting out the excess pressure inside the primary balloon.

According to another embodiment of the catheter, wherein the restraining mechanism is a restraining balloon smaller than the primary balloon, and placed onto a part of the primary balloon, so as to enclose a part of the primary balloon, such that the enclosed part of the primary balloon makes the first part of the primary balloon, the restraining balloon has a stretchable opening which keeps the first part of the balloon and the second part of the balloon incommunicado till the time pressure inside the second part surpasses the first predefined threshold. This embodiment provides another efficient way to implement the restraining mechanism for dividing the primary balloon into two parts.

According to yet another embodiment of the catheter, wherein the restraining mechanism is a restraining valve which is adapted to be in a closed position to keep the first part of the primary balloon or the opening of the primary balloon in the restrained position until the pressure inside the second part of the primary balloon or the primary balloon is within the first predefined threshold, and to be in a open position to keep the first part of the primary balloon or the opening of the primary balloon in the extended position when the pressure inside the second part of the primary balloon or the primary balloon surpasses the first predefined threshold, and until the pressure inside the second part of the primary balloon or the primary balloon gets below the first predefined threshold. This embodiment provides one more efficient way to implement the restraining mechanism.

According to one embodiment of the catheter, wherein the inflation/deflation port is placed at a proximal end of the catheter and the primary balloon is placed in proximity to a distal end of the catheter.

### BRIEF DESCRIPTION OF THE DRAWINGS

FIG. 1 illustrates an exemplary modified foley's catheter having stretchable band to divide a primary balloon into two parts.

FIG. 2 illustrates another exemplary modified foley's catheter having stretchable band placed in between an opening of the primary balloon to an environment outside of the primary balloon.

FIG. 3 illustrates yet another exemplary modified foley's catheter having a fluid communication opening between a primary balloon and secondary balloon checked through a check valve.

FIG. 4 illustrates yet another exemplary modified foley's catheter having a magnetic valve placed onto a fluid communication channel of the first part of the primary balloon and an outside environment.

FIG. 5 illustrates yet another exemplary modified foley's catheter having a fluid communication opening in the first part of the primary balloon.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### DESCRIPTION

For the purpose of promoting an understanding of the principles of the invention, reference will now be made to the embodiment illustrated in the figures and specific language will be used to describe them. It will nevertheless be understood that no limitation of the scope of the invention is thereby intended. Such alterations and further modifications in the illustrated system, and such further applications of the principles of the invention as would normally occur to those skilled in the art are to be construed as being within the scope of the present invention.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

Embodiments of the present invention will be described below in detail with reference to the accompanying figure.

As already discussed in the Background, there are adverse effects of conventional Foley's catheter. Similar adverse effect can be found in any of the catheters having balloons and which are used inside the human body to be inflated or deflated to retain the balloon inside the human body part or to apply a medication inside the human body part, or to install a device inside the human body part.

These adverse effects can be checked through pressure induced deflations techniques onto the balloon of the catheter. Some of the techniques discussed are as follows.

In first embodiment, a primary balloon of the catheter can be divided into two parts, a first part and a second part. The second part shall be in direct fluid communication with a pumping means through an inflation/deflation port. The pumping means is connected to the inflation/deflation port so as to pump in a working fluid to inflate the primary balloon, and to pump out the fluid to deflate the primary balloon. The primary balloon is divided into two parts using a stretchable band which cover a part of the primary balloon wherein the second part is inflated using the pumping means for retaining the second part inside a human body part or to apply a medication inside the human body part, or to install a device inside the human body part. The stretchable band keeps the first part of the primary balloon in a restrained position until a pressure inside the second part is within a first predefined threshold. This helps in keeping the fluid communication closed between the first part and the second part of the primary balloon until a first predefined threshold is reached. Further, when the pressure inside the second part surpasses the first predefined threshold, the stretchable band stretches to allow fluid communication from the second part to the first part of the primary balloon, so that the first part moves into an expanded position.

It is to be noted that the first predefined threshold of pressure is much lesser than the pressure required to traumatize/injure a lumen or body cavity or similar parts of human body where the primary balloon shall be placed or put into traction. This first predefined threshold pressure can differ from person to person, and one body part to another body part. Accordingly, the stretchable capabilities of the stretchable bands can differ based on its usage for different human being and for different body parts.

This implementation clearly helps in keeping the check on expansion of the second part of the balloon, so that the second part's expansions shall not bring adverse effect on the human body part where the primary balloon is placed.

In one additional implementation of this first embodiment, further checks for the pressure built up in the first part of the primary balloon is provided. It is to be noted that in case the pressure keeps on building in the first part, it can again be harmful for a human body part where the primary balloon is placed. Hence, there should be certain mechanisms which can help in checking out the pressure built up in the first part of balloon also, and these checking mechanisms gets activated when the pressure built up in the first part surpasses a second predefined pressure threshold.

One of the above-mentioned checking mechanisms can be implemented through provision of a weak area inside the first part of the primary balloon. This weak area can be provided either onto external layer of the first part of the primary balloon which provides venting out of the working fluid into lumen/cavity of the body part where the primary balloon is placed, or onto an internal layer which is in physical coupling to a working fluid channel through which the first part of the primary balloon is inflated/deflated using the working fluid or a body fluid drainage channel through which a body fluid is drained out of the human body. Once the pressure inside the first part of the primary balloon is beyond the second predefined threshold, this weak area bursts to vent out the fluid.

It is to be noticed that second predefined pressure threshold shall be equal to or lesser than the first predefined pressure threshold.

Another way to provide a check to pressure built up inside the first part of the primary balloon can be implemented through a provision of a flow controller which opens a fluid communication channel between the first part of the primary balloon and the environment outside of the primary balloon when a pressure inside the first part of the primary balloon surpasses the second predefined threshold, and to close the fluid communication channel between the first part of the primary balloon and the environment outside of the primary balloon when the pressure inside the first part of the primary balloon gets below the second predefined threshold. The environment outside the primary balloon can be a working fluid channel through which the primary balloon is inflated/deflated using the working fluid, or a body fluid drainage channel through which a body fluid is drained out of the human body, or lumen/cavity of the body part in which the primary balloon of the catheter is placed, or a secondary balloon connected to the first part of the primary balloon through the fluid communication channel via the restraining mechanism.

In exemplary implementation of the flow controller, the flow controller can be an automated valve or a retractable plug. The automated valve moves on or off the fluid communication channel for opening or closing the fluid communication channel. While, the retractable plug unplugs itself from the fluid communication channel or retract back to plug itself onto the fluid communication channel for opening or closing the fluid communication channel respectively. The automated valve or the retractable plug works on at least one of the mechanical, electrical or magnetic principles, or combination thereof.

It is to be noted that no external stretchable band is required in one alternate implementation of the first embodiment, rather a particular area of the primary balloon itself is made with a different stretchability with respect to the other area of the primary balloon, and makes this area of the primary balloon suitable to act as a stretchable band.

In a second embodiment, the stretchable band is placed at an opening of the primary balloon which connects the primary balloon to the environment outside of the primary balloon. The environment outside the primary balloon can be a working fluid channel through which the primary balloon is inflated/deflated using the working fluid, or a body fluid drainage channel through which a body fluid is drained out of the human body, or a cavity/lumen of the body part in which the primary balloon of the catheter is placed, or a secondary balloon connected to the primary balloon through the fluid communication channel via the restraining mechanism. As the pressure builds up inside the primary balloon and goes beyond the first predefined threshold, the stretchable band expands and the opening of the primary balloon moves in an expanded position allowing the working fluid to move out of the primary balloon to the external environment. Further, when the pressure inside the primary balloon reduces and gets below the first predefined threshold, the stretchable band contracts, and the opening of the primary balloon moves into the restrained position.

In variations from the first embodiment and the second embodiment, instead of the stretchable band, any other restraining mechanism which can provide similar functionality of the stretchable band can be provided. This means to restrain the first part of the primary balloon or the opening of the primary balloon whenever pressure remains below the first predefined threshold inside the second part of primary balloon or the primary balloon, and to expand the first part of the primary balloon or the opening of the primary balloon whenever pressure surpasses the first predefined threshold inside the second part of the primary balloon or the primary balloon. One such restraining mechanism can be a restraining valve which is adapted to be in a closed position to keep the first part of the primary balloon or the opening of the primary balloon in the restrained position until the pressure inside the second part of the primary balloon or the primary balloon is within a first predefined threshold, and to be in an open position to keep the first part of the primary balloon or the opening of the primary balloon in the extended position when the pressure inside the second part of the primary balloon or the primary balloon surpasses the first predefined threshold.

In the third embodiment, which is a close alternative to the first embodiment, a restraining balloon provides the same functionality as the stretchable band. The restraining balloon is smaller than the primary balloon. The restraining balloon is placed onto a part of the primary balloon, so as to enclose a part of the primary balloon. The enclosed part of the primary balloon makes the first part of the primary balloon. The restraining balloon has a stretchable opening which keeps the first part of the primary balloon and the second part of the primary balloon incommunicado till the time pressure inside the second part surpasses the first predefined threshold.

The invention is further described using various figures, which proposes a modified Foley's catheter. Even though, the implementation is explained through modified Foley's catheters, same features can be implemented in any other type of catheters which uses inflatable balloons inside human body.

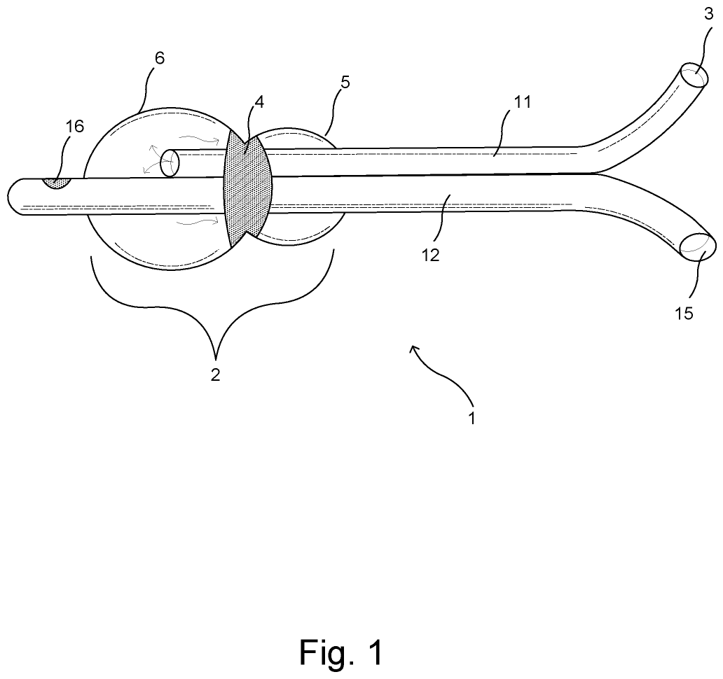

FIG. 1 illustrates an exemplary modified foley's catheter 1 having stretchable band 4 which divides a primary balloon 2 into two parts, a first part 5 and a second part 6. The foley's catheter has a working fluid channel 11 and a body fluid drainage channel 12. The working fluid channel 11 has an inflation/deflation port 3 through which the working fluid is infused to inflate the primary balloon 2 or the working fluid is removed to deflate the primary balloon 2. The inflation/deflation port 3 can be connected to a pumping means to pump in the working fluid into the primary balloon 2 through the working fluid channel 11 or to pump out the working fluid out of the primary balloon 2 to deflate the primary balloon 2. The purpose of inflation of the primary balloon 2 is to expand the primary balloon 2 sufficiently, so as to retain the primary balloon 2 inside the bladder of a patient. The body fluid drainage channel 12 extends from distal end (tip of the catheter) to the proximal end of the catheter 1 through two openings 15, 16. The proximal opening 15 of the body fluid channel 12 is provided in proximity to the proximal end of the catheter 1 through which the body fluid moves out of the body fluid drainage channel 12 to an external environment. The distal opening 16 of the body fluid drainage channel of the catheter 1 is provided in proximity to the distal end of the body fluid drainage channel 12 to drain out the body fluid into the body fluid drainage channel 12. Firstly, the second part 6 of the primary balloon 2 is filled with the working fluid through the working fluid channel. In case, if excess fluid is filled inside the second part 6 and the pressure builds up inside the second part 6 exceeds beyond a first predefined threshold, the stretchable band 4 expands and allow excess fluid to move into the first part 5 of the primary balloon 2. Such excess pressure builds up beyond the predefined threshold can happen due to any excess resistance encountered by the second part 6 of the primary balloon 1, or the primary balloon 1 itself, either due to excessive inflation of the retaining balloon in a lumen like urethra/blood vessel or forced pull out/traction of the catheter 1, and in such scenario also the stretchable band 4 can be expanded. In furtherance, when the pressure inside the second part 6 is released and moves back within the first predefined threshold, the stretchable band 4 contracts to close fluidic communication between the second part 6 and the first part 5. This helps to keep the pressure inside the primary balloon 2 always within the safety bearable limits for the human body part.

As further embodiments explained through figures are also based on foley's catheter, hence only the modified features shall be discussed to keep the discussion concise. Even though reference numerals may be provided for all features in the figures, however, each of the features are not discussed, and there structural and functional understanding are similar as discussed in FIG. 1.

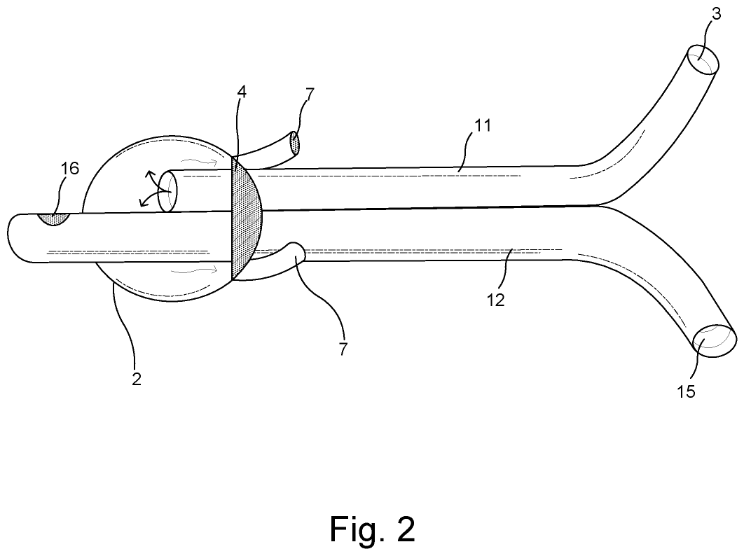

FIG. 2 illustrates another exemplary modified foley's catheter having stretchable band 4 placed between an opening 7 of the primary balloon 2 to an environment outside of the primary balloon 2. In this embodiment, instead of dividing the primary balloon 4 into two parts, the stretchable band 4 is placed on opening 7 of the primary balloon 2 which connects into cavity/lumen of the body part and the body fluid drainage channel 12. Accordingly, when the pressure builds up and exceeds beyond the first predefined threshold inside the primary balloon 2, the stretchable band 4 expands, so as to vent out the excess working fluid into cavity/lumen of the human body part and the body fluid drainage channel 12. When the pressure inside the primary balloon 2 revert back within the first predefined threshold, the stretchable band 4 contracts and restrains the opening 7 of the primary balloon 2 to close fluid communication to the cavity/lumen of the body part and the body fluid drainage channel 12.

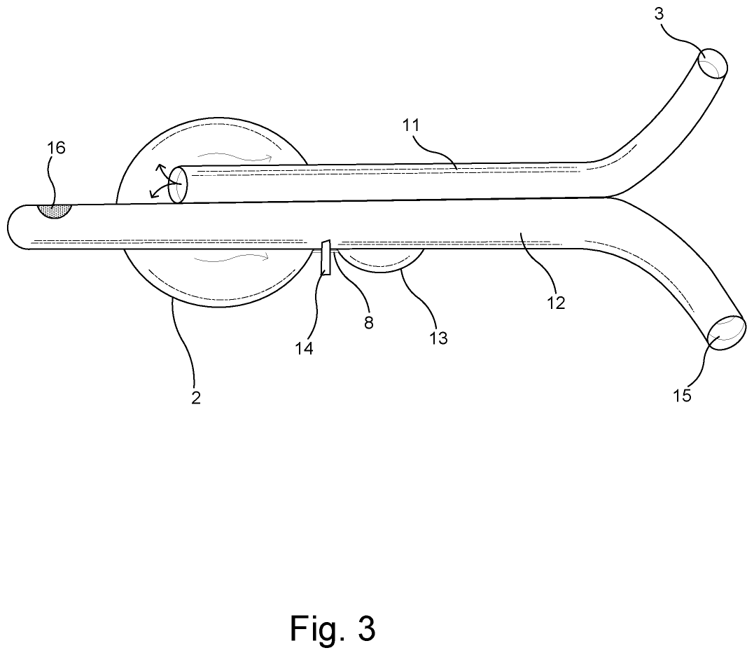

FIG. 3 illustrates yet another exemplary modified foley's catheter 1 having a fluid flow channel 8 between a primary balloon 2 and secondary balloon 13 checked through a check valve 14. The secondary balloon 13 is placed in proximity to primary balloon 2, and shall remain inside the human body when the catheter is placed inside the human body. In this embodiment, no stretchable band is provided, rather check valve 14 is provided to implement the functionality of restraining mechanism. When the excess pressure is built up inside the primary balloon 2, and it surpasses the first predefined threshold, the check valve 14 opens up and allow the working fluid to flow from the primary balloon 2 to the secondary balloon 13 to normalize the pressure inside the primary balloon 2. When the pressure inside the primary balloon 2 gets within the predefined threshold, the check valve 14 closes the fluid communication between the primary balloon 2 and the secondary balloon 13.

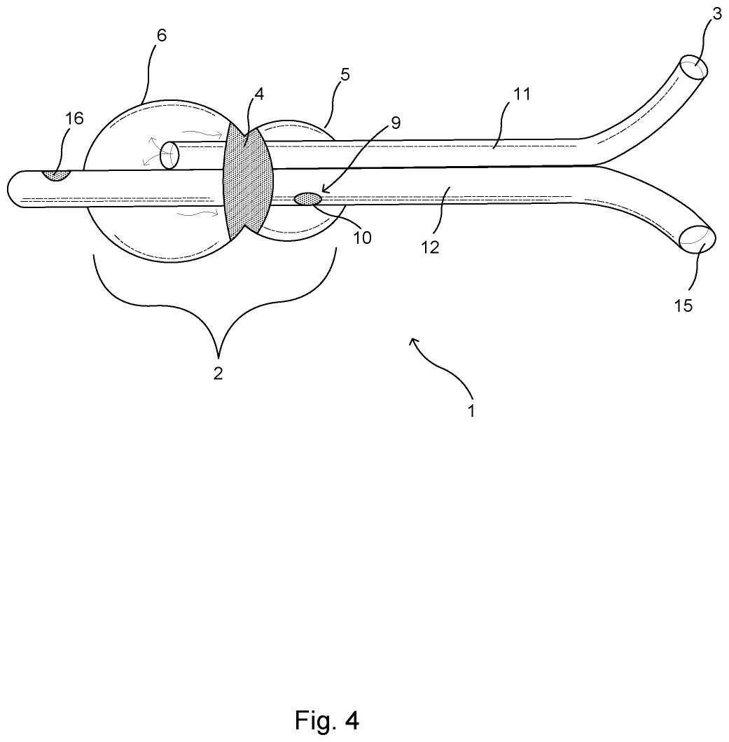

FIG. 4 illustrates yet another exemplary modified foley's catheter 1 having an automated valve 10, which works on magnetic principles, placed onto a fluid communication opening 9 of the first part 5 of the primary balloon 2 and the body fluid drainage channel 12. This embodiment is an enhancement of the embodiment provided in FIG. 1. This feature is provided to make the catheter 1 further safe, so that the excess pressure does not build up inside the first part 5 and creates a problem for the body part. This implementation provides to vent out excess fluid which is creating excess pressure build up in the first part 5 of the primary balloon 2. Here, the automated valve 10 is placed onto the fluid communication opening 9 formed between an internal surface of the primary balloon 1 over catheter shaft region and the body fluid drainage channel 12. This automated valve 10 works on the magnetic principle. The magnet inside the automated valve can be mechanically driven or electrically driven. In this embodiment the magnet is mechanically driven such that the automated valve 10 is retractably placed, and when the pressure inside the first part 5 of the primary balloon builds up and exceeds beyond a second predefined threshold, the force applied due to the excess working fluid goes beyond the magnetic force of the automated valve 10, and pushes the automated valve 10 to vent out the excess working fluid into the body fluid drainage channel 12. When the excess fluid moves out, the pressure inside the first part 5 of the primary balloon 2 reduces, and according the force applied by the working fluid inside the first part 5 also reduces and gets lesser than the magnetic force, and accordingly, the automated valve retracts back onto the fluid communication opening 9 to close the communication between the first part 5 of the primary balloon 2 and the body fluid drainage channel 12.

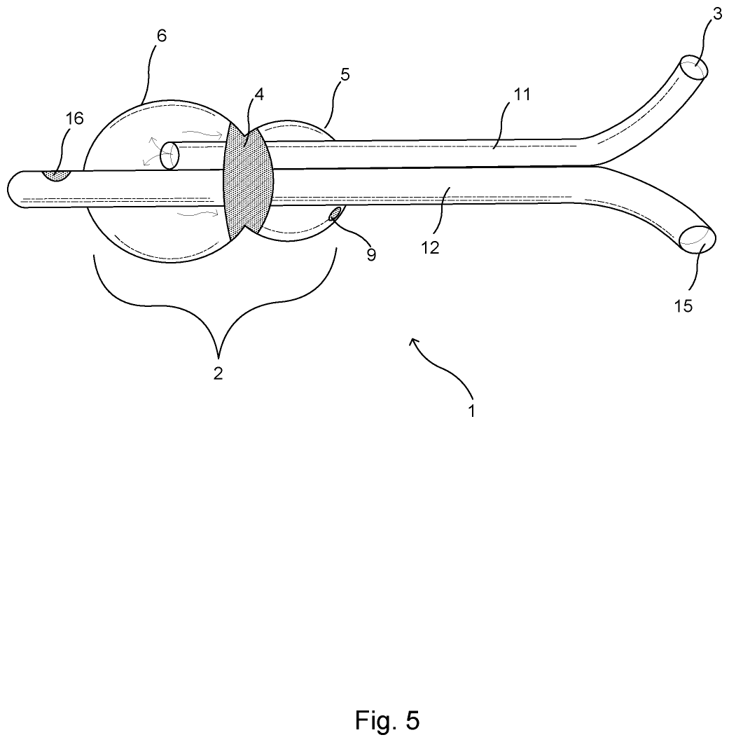

FIG. 5 illustrates an exemplary modified foley's catheter as in FIG. 1 having stretchable band 4 which divides a primary balloon 2 into two parts, a first part 5 and a second part 6. There is a fluid communication opening 9 in the outer surface of the first part 5 of the primary balloon 2 to vent out the working fluid promptly to the environment outside the catheter 1 i.e., into the cavity or lumen of the human body part where the catheter 1 is applied. Firstly, the second part 6 of the primary balloon 2 is filled with the working fluid through the working fluid channel 11 for retention of the catheter 1 inside the urinary bladder. In case, if the pressure build up inside the second part 6 of the primary balloon 2 exceeds beyond a first predefined threshold due to excessive inflation of second part 6 of the primary balloon 2 with working fluid or due to increasing resistance from the body part resulting from traction of the catheter 1 leads to the expansion of the stretchable band 4 allowing the fluid to move into the first part 5 of the primary balloon 2 and further into environment outside the first part 5 of the primary balloon 2 through the fluid communication opening 9 in the outer surface of the first part 5 of the primary balloon 2 which may be noticed by the administrator as a peri-catheter leak of the working fluid. This working fluid which has been vented out from the second part 6 of the primary balloon 2 into the first part 5 will be expelled into the outside environment of the first part 5 of the primary balloon 2 to maintain the first part 5 of the primary balloon 2 in a collapsed state. In furtherance, when the pressure inside the second part 6 is vented out to an extent below the first predefined threshold, the stretchable band 4 contracts to close fluidic communication between the second part 6 and the first part 5. This keeps the pressure inside the primary balloon 2 always within the safety bearable limits for the human body part.

It is to be noted that the “fluid communication channel” discussed in the description has both conceptual as well as physical connotation, as the case may be. It can represent as a flow of fluid from one side to another to depict conceptual connotation, or it can depict a tube like structure or a hole like structure which allows flow of fluid from one side to another.

Paradoxically the above invention can be utilized to remove non-deflatable catheter (due manufacturing defect or occasional inflation of the balloon with normal saline or similar solution which crystallizes in the balloon channel thus preventing deflation) safely with a gentle gradual traction on the catheter that will result in the catheter expulsion and thus avoiding/minimizing radiology consultations/endoscopic intervention.

The above mentioned methods may be utilized alone or in various combinations or with mild modifications/similar rearrangements resulting in pressure induced deflation of the catheter as the final end point and thus in devising a catheter which may minimize or totally avoid the inflated catheter induced urethral trauma and its attendant complications secondary to inadvertent pulling (catheter traction induced urethral injury) of the catheter (by the patient)/inflation of the catheter in the urethra (by the health care personnel).

### LIST OF REFERENCE SIGNS

1 Catheter
2 Primary Balloon
3 Inflation/deflation port
4 Stretchable band
5 First part of the primary balloon
6 Second part of the primary balloon
7 Opening of the primary balloon
8 Fluid flow channel
9 Fluid communication opening
10 Automated valve
11 Working fluid channel
12 Body fluid drainage channel
13 Secondary balloon
14 Check valve
15 Proximal opening of the body fluid drainage channel of the Catheter
16 Distal opening of the body fluid drainage channel of the Catheter

I/We claim:

1. A catheter (1) comprising:
a primary balloon (2) adapted to be inflated or deflated to retain the primary balloon (2) inside a human body part or to apply a medication inside the human body part, or to install a device inside the human body part,
a inflation/deflation port (3) adapted to be connected to a pumping means to pump in a working fluid to inflate the primary balloon (2), and to pump out the fluid to deflate the primary balloon (2),
a restraining mechanism adapted:
to keep a first part (5) of the primary balloon (2) or an opening (7) of the primary balloon (2) in a restrained position until a pressure inside the second part (6) of the primary balloon (2) or the primary balloon (2) is within a first predefined threshold, so as to close a fluid communication:
between a second part (6) of the primary balloon (2) and the first part (5) of the primary balloon (2), or
between the opening (7) of the primary balloon (2) and an environment outside of the primary balloon (2), and
to keep the first part (5) of the primary balloon (2) or the opening (7) of the primary balloon (2) in an expanded position when the pressure inside the second part (6) of the primary balloon (2) or the primary balloon (2) surpasses the first predefined threshold and until the pressure inside the second part (6) of the primary balloon (2) or the primary balloon (2) gets below the first predefined threshold, so as to open a fluid communication:
between the second part (6) of the primary balloon (2) and the first part (5) of the primary balloon (2), or
between the opening (7) of the primary balloon (2) and the environment outside of the primary balloon (2).

2. The catheter as claimed in claim 1, wherein the restraining mechanism is a stretchable band (4) which is adapted to:
cover a part of the primary balloon (2) dividing the primary balloon (2) into the first part (5) and the second part (6), wherein the second part (6) is adapted to be inflated or deflated using the pumping means for retaining the second part (6) of the primary balloon (2) inside a human body part or to apply a medication inside the human body part, or to install a device inside the human body part, or
placed at the opening (7) of the primary balloon (2) which connects the primary balloon (2) to the environment outside of the primary balloon (2).

3. The catheter (1) as claimed in claim 2, wherein the first part (5) of the primary balloon (2) comprises a weak area which is adapted to burst when a pressure inside the first part (5) of the primary balloon (2) surpasses a second predefined threshold.

4. The catheter (1) as claimed in claim 2, wherein the first part (5) of the primary balloon (2) and the environment outside of the primary balloon (2) are fluidly connected to each other through a fluid communication channel, the catheter (1) comprises a flow controller which is adapted to open the fluid communication channel between the first part (5) of the primary balloon (2) and the environment outside of the primary balloon (2) when a pressure inside the first part (5) of the primary balloon (2) surpasses the second predefined threshold, and to close the fluid communication channel between the first part (5) of the primary balloon (2) and the environment outside of the primary balloon (2) when the pressure inside the first part (5) of the primary balloon (2) gets below the second predefined threshold.

5. The catheter (1) as claimed in claim 4, wherein the flow controller is an automated valve or a retractable plug, wherein the automated valve is adapted to move on or off the fluid communication channel for opening or closing the fluid communication channel, and the retractable plug is adapted to unplug itself from the fluid communication channel or retract back to plug itself onto the fluid communication channel for opening or closing the fluid communication channel, wherein the automated valve or the retractable plug works on at least one of the mechanical, electrical or magnetic principles, or combination thereof.

6. The catheter (1) as claimed in claim 2, wherein the environment outside the primary balloon (2) is:
a working fluid channel (11) through which the primary balloon (2) is inflated using the working fluid, or
a body fluid drainage channel (12) through which a body fluid is drained out of the human body, or
a cavity/lumen of the body part in which the primary balloon (2) of the catheter (1) is placed, or
a secondary balloon (13) connected to the primary balloon (2) through a fluid flow channel (8) via the restraining mechanism, wherein the secondary balloon (13) is placed in proximity to the primary balloon (2).

7. The catheter (1) as claimed in claim 1, wherein the restraining mechanism (4) is a restraining balloon smaller than the primary balloon (2), and placed onto a part of the primary balloon (2), so as to enclose a part of the primary balloon (2), such that the enclosed part of the primary balloon (2) makes the first part (5) of the primary balloon (2), the restraining balloon has a stretchable opening which keeps the first part (5) of the primary balloon (2) incommunicado to the second part (6) of the primary balloon (2) till the time pressure inside the second part (6) surpasses the first predefined threshold.

8. The catheter (1) as claimed in claim 1, wherein the restraining mechanism (4) is a restraining valve which is adapted to be in a closed position to keep the first part (5) of the primary balloon (2) or the opening (7) of the primary balloon (2) in the restrained position until the pressure inside the second part (6) of the primary balloon (2) or the primary balloon (2) is within a first predefined threshold, and to be in an open position to keep the first part (5) of the primary balloon (2) or the opening (7) of the primary balloon (2) in the extended position when the pressure inside the second part (6) of the primary balloon (2) or the primary balloon (2) surpasses the first predefined threshold, and until the pressure inside the second part (6) of the primary balloon (2) or primary balloon (2) gets below the first predefined threshold.

9. The catheter (1) as claimed in claims 1 to 8, wherein the inflation/deflation port (3) is placed at a proximal end of the catheter (1) and the primary balloon (2) is placed in proximity to a distal end of the catheter (1).
